(19) United States
(12) Patent Application Publication
(10) Pub. No.: US 2025/0354115 A1
(43) Pub. Date: Nov. 20, 2025

(54) METHOD FOR PRODUCING STEM CELLS AND METHOD FOR PRODUCING GAMMA DELTA T CELLS

(71) Applicants: Koji Tanabe, Palo Alto, CA (US); I Peace, Inc., Palo Alto, CA (US)

(72) Inventors: Koji TANABE, Palo Alto, CA (US); Kenta SUTO, Kyoto (JP); Yutaka YASUI, Palo Alto, CA (US)

(73) Assignees: Koji Tanabe, Palo Alto, CA (US); I Peace, Inc., Palo Alto, CA (US)

(21) Appl. No.: 18/868,650

(22) PCT Filed: May 9, 2023
(86) PCT No.: PCT/JP2023/017460

§ 371 Date: Nov. 22, 2024

Related U.S. Application Data
(60) Provisional application No. 63/344,860, filed on May 23, 2022.
(60) Provisional application No. 63/492,196, filed on Mar. 24, 2023.

(51) Int. Cl. C12N 5/0783; A61K 35/17

(52) U.S. Cl. CPC C12N 5/0636; A61K 35/17; C12N 2500/42; C12N 2506/45

(57) ABSTRACT

According to the present disclosure, a method for producing stem cells is provided, comprising applying tetrakis-pivaloyloxymethyl 2-(thiazol-2-ylamino) ethylidene-1, 1-bisphosphonate (PTA) to blood cells or immune cells, and inducing stem cells from said blood cells or immune cells.

### TECHNICAL FIELD

The present invention relates to cell technology and specifically to a method for producing stem cells and a method for producing γδ T cells.

### BACKGROUND ART

T cells derived from hematopoietic stem cells play a crucial role in the immune system (see, for example, Non-Patent Document 1). T cells express a highly diverse T-cell receptor (TCR) on their surface. The diversity of TCRs is created by V (D) J gene recombination. FIG. 1 shows the VJ gene recombination process.

DN1 (double negative 1) cells, which are precursor cells of T cells, proliferate in response to interleukin-7 (IL-7) and c-kit ligand (KL), which are strongly expressed in the subcapsular region of the thymic cortex. DN1 cells then express CD25 (interleukin-2 receptor a chain) and differentiate into DN2 cells. DN2 cells gradually reduce the expression of CD44 and further differentiate into DN3 cells.

TCRs include a variable (V) region and a constant (C) region. The amino acid sequence of the V region is highly diverse and forms the antigen-binding site. In germline DNA, the V region genes are separated into V genes, D (diversity) genes, and J (joining) genes. These genes are recombined on the chromosome during the process of T cell differentiation, forming a continuous V-(D)-J structure, which becomes an expressed gene. The J genes include JP1, JP, J1, JP2, and J2 genes.

DN3 cells, in which V (D) J gene recombination has occurred at the TCRβ and TCRγ gene loci, differentiate into either TCRαβ-type T cells or TCRγδ-type T cells. In TCRαβ-type T cells, the TCR is composed of an α-chain and a β-chain, whereas in TCRγδ-type T cells, the TCR is composed of a γ-chain and a δ-chain. The determination to become either TCRαβ-type or TCRγδ-type is controlled by regulatory elements in the silencer regions of the TCRγ and TCRα gene loci.

Although TCRγδ-type T cells are fewer in number than TCRαβ-type T cells, they are predominant among intraepithelial lymphocyte populations in the intestinal mucosa. TCRγδ-type T cells can sense various stresses that cause cellular damage and induce immune responses. It is believed that TCRγδ-type T cells can detect not only external stresses, such as bacterial or viral infections, but also changes in the properties of cells that accompany cancer development.

TCRγδ-type T cells proliferate and become activated after recognizing intermediates of the mevalonate pathway in the cholesterol synthesis of antigen-presenting cells (APCs) or isopentenyl pyrophosphate (IPP) as antigens. Accordingly, cancer immunotherapy involving the activation of a patient's TCRγδ-type T cells ex vivo and their reinfusion into the body has been implemented. However, TCRγδ-type T cells constitute only 1-5% of peripheral blood, making it difficult to obtain a sufficient number of TCRγδ-type T cells from blood samples.

As a result, methods have been proposed to induce iPS cells with recombined γδ-TCR genes by reprogramming peripheral blood mononuclear cells (PBMCs) stimulated by zoledronic acid (see, for example, Patent Document 1). However, it has been reported that the γδ-TCR genes induced by this method do not undergo recombination of J1/J2 genes (see, for example, Non-Patent Document 2).

### PATENT DOCUMENTS

[Patent Document 1] International Publication No. WO 2018/143243

### NON-PATENT LITERATURE

[Non-Patent Document 1] Shoichi Iriguchi et al., “A clinically applicable and scalable method to regenerate T-cells from iPSCs for off-the-shelf T-cell immunotherapy,” NATURE COMMUNICATIONS, 2021, 12:430

[Non-Patent Document 2] DAISUKE WATANABE et al., “The Generation of Human γδ T Cell-Derived Induced Pluripotent Stem Cells from Whole Peripheral Blood Mononuclear Cell Culture,” STEM CELLS TRANSLATIONAL MEDICINE, 2018; 7:34-44

### SUMMARY OF THE INVENTION

### Problem to be Solved by the Invention

One of the objectives of the present invention is to provide a method for efficiently inducing stem cells with recombined γδ-TCR genes, as well as a method for efficiently producing γδ T cells.

### Means for Solving the Problem

A method for producing stem cells according to one aspect of the present invention includes applying tetrakis-pivaloyloxymethyl 2-(thiazol-2-ylamino) ethylidene-1, 1-bisphosphonate (PTA) to blood cells or immune cells, and inducing stem cells from the blood cells or immune cells.

The above method for producing stem cells may further include applying interleukin to the blood cells or immune cells.

In the above method for producing stem cells, the interleukin may be at least one selected from the group consisting of IL-2, IL-4, IL-9, IL-18, and IL-33.

In the above method for producing stem cells, the blood cells or immune cells may be mononuclear cells.

In the above method for producing stem cells, the stem cells may be induced pluripotent stem (iPS) cells.

In the above method for producing stem cells, and in the induction of the stem cells from the blood cells or immune cells, an induction factor RNA may be introduced into the blood cells or immune cells.

In the above method for producing stem cells, and in the induction of stem cells from the blood cells or immune cells, an RNA virus vector may be used. The RNA virus vector may be a single-stranded RNA virus vector. The RNA virus vector may be a single-stranded positive RNA virus vector. The RNA virus vector may be a single-stranded negative RNA virus vector. The RNA virus vector may be a non-integrating RNA virus vector. The RNA virus vector may be a Mononegavirales order virus vector. The RNA virus vector may be a Paramyxoviridae family virus vector. The RNA virus vector may be a Respirovirus genus virus vector. The RNA virus vector may be a Sendai virus vector.

In the above method for producing stem cells, and in the induction of stem cells from the blood cells or immune cells, a chimeric virus that comprises viral genomic RNA harboring the induction factor RNA and an envelope encompassing the genomic RNA and derived from a virus different from that of the genomic RNA may be used.

In the above method for producing stem cells, the stem cells may have recombined γδ-TCR genes.

A method for producing blood cells or immune cells according to another aspect of the present invention comprises preparing the stem cells produced by the above method for producing stem cells, and inducing blood cells or immune cells from the stem cells.

In the above method for producing blood cells or immune cells, the blood cells or immune cells may be γδ T cells.

In the above method for producing blood cells or immune cells, and in the induction of the blood cells or the immune cells from the stem cells, a cell mass of the stem cells may be seeded onto feeder cells.

In the above method for producing blood cells or immune cells, the feeder cells may be stromal cells.

A method for producing stem cells according to one aspect of the present invention includes applying bisphosphonate or (E)-4-hydroxy-3-methyl-but-2-enyl pyrophosphate (HMBPP), and interleukin to blood cells or immune cells, and inducing stem cells from the blood cells or immune cells, wherein the interleukin is at least one selected from the group consisting of IL-4, IL-9, IL-18, and IL-33, and the interleukin may exclude IL-2.

In the above method for producing stem cells, the interleukin may include IL-18 and IL-33.

In the above method for producing stem cells, the interleukin may include IL-4 and IL-18.

In the above method for producing stem cells, the interleukin may include IL-9 and IL-18.

In the above method for producing stem cells, the interleukin may include IL-4, IL-9, and IL-18.

In the above method for producing stem cells, the interleukin may include IL-4, IL-18, and IL-33.

In the above method for producing stem cells, IL-4 may be applied after applying IL-18 and IL-33 to the blood cells or immune cells.

In the above method for producing stem cells, the interleukin may include IL-4, IL-9, IL-18, and IL-33.

In the above method for producing stem cells, IL-4 may be applied to the blood cells or immune cells after applying IL-9, IL-18, and IL-33 to the blood cells or immune cells.

In the above method for producing stem cells, the bisphosphonate may be selected from zoledronic acid, pamidronic acid, alendronic acid, risedronic acid, ibandronic acid, incadronic acid, etidronic acid, minodronic acid, tetrakis-pivaloyloxymethyl 2-(thiazol-2-ylamino) ethylidene-1, 1-bisphosphonate (PTA), salts thereof, and hydrates thereof.

In the above method for producing stem cells, the bisphosphonate may be zoledronic acid.

In the above method for producing stem cells, the blood cells or immune cells may be mononuclear cells. In the above method for producing stem cells, the stem cells may be induced pluripotent stem (iPS) cells.

In the above method for producing stem cells, and in the induction of stem cells from the blood cells or immune cells, an induction factor RNA may be introduced into the blood cells or immune cells.

In the above method for producing stem cells, and in the induction of stem cells from the blood cells or immune cells, an RNA virus vector may be used. The RNA virus vector may be a single-stranded RNA virus vector. The RNA virus vector may be a single-stranded positive RNA virus vector. The RNA virus vector may be a single-stranded negative RNA virus vector. The RNA virus vector may be a non-integrating RNA virus vector. The RNA virus vector may be a Mononegavirales order virus vector. The RNA virus vector may be a Paramyxoviridae family virus vector. The RNA virus vector may be a Respirovirus genus virus vector. The RNA virus vector may be a Sendai virus vector.

In the above method for producing stem cells, and in the induction of stem cells from the blood cells or immune cells, a chimeric virus comprising viral genomic RNA harboring the induction factor RNA, and an envelope encompassing the genomic RNA and derived from a virus different from that of the genomic RNA may be used.

In the above method for producing stem cells, the stem cells may have recombined γδ-TCR genes.

A method for producing blood cells or immune cells according to another aspect of the present invention comprises preparing the stem cells produced by the above method for producing stem cells and inducing blood cells or immune cells from the stem cells.

In the above method for producing blood cells or immune cells, the blood cells or immune cells may be γδ T cells.

In the above method for producing blood cells or immune cells, and in the induction of the blood cells or the immune cells from the stem cells, a cell mass of the stem cells may be seeded onto feeder cells.

In the above method for producing blood cells or immune cells, the feeder cells may be stromal cells.

A method for producing stem cells according to another aspect of the present invention comprises applying bisphosphonate or (E)-4-hydroxy-3-methyl-but-2-enyl pyrophosphate (HMBPP) to blood cells or immune cells, and inducing stem cells from the blood cells or immune cells, wherein IL-2 is not applied to the blood cells or immune cells. Preferably, any interleukin that includes IL-2 is not applied to the blood cells or immune cells.

In the above method for producing stem cells, the bisphosphonate may be selected from zoledronic acid, pamidronic acid, alendronic acid, risedronic acid, ibandronic acid, incadronic acid, etidronic acid, minodronic acid, tetrakis-pivaloyloxymethyl 2-(thiazol-2-ylamino) ethylidene-1, 1-bisphosphonate (PTA), salts thereof, and hydrates thereof.

In the above method for producing stem cells, the bisphosphonate may be zoledronic acid.

In the above method for producing stem cells, the blood cells or immune cells may be mononuclear cells.

In the above method for producing stem cells, the stem cells may be induced pluripotent stem (iPS) cells.

In the above method for producing stem cells, and in the induction of the stem cells from the blood cells or immune cells, an induction factor RNA may be introduced into the blood cells or immune cells.

In the above method for producing stem cells, and in the induction of the stem cells from the blood cells or immune cells, an RNA virus vector may be used. The RNA virus vector may be a single-stranded RNA virus vector. The RNA virus vector may be a single-stranded positive RNA virus vector. The RNA virus vector may be a single-stranded negative RNA virus vector. The RNA virus vector may be a non-integrating RNA virus vector. The RNA virus vector may be a Mononegavirales order virus vector. The RNA virus vector may be a Paramyxoviridae family virus vector. The RNA virus vector may be a Respirovirus genus virus vector. The RNA virus vector may be a Sendai virus vector.

In the above method for producing stem cells, and in the induction of stem cells from the blood cells or immune cells, a chimeric virus comprising viral genomic RNA harboring the induction factor RNA, and an envelope encompassing the genomic RNA and derived from a virus different from that of the genomic RNA may be used.

In the above method for producing stem cells, the stem cells may have recombined γδ-TCR genes.

A method for producing blood cells or immune cells according to an embodiment of the present invention includes preparing stem cells produced by the above method for producing stem cells and inducing blood cells or immune cells from the stem cells.

In the above method for producing blood cells or immune cells, the blood cells or immune cells may be γδ T cells.

In the above method for producing blood cells or immune cells, and in the induction of blood cells or immune cells from the stem cells, a cell mass of the stem cells may be seeded onto feeder cells.

In the above method for producing blood cells or immune cells, the feeder cells may be stromal cells.

A method for producing stem cells according to an embodiment of the present invention includes applying tetrakis-pivaloyloxymethyl 2-(thiazol-2-ylamino) ethylidene-1, 1-bisphosphonate (PTA) to blood cells or immune cells and inducing stem cells from the blood cells or immune cells, wherein IL-2 is not applied to the blood cells or immune cells.

The above method for producing stem cells may further include applying interleukin other than IL-2 to the blood cells or immune cells.

In the above method for producing stem cells, the interleukin may be at least one selected from the group consisting of IL-4, IL-9, IL-18, and IL-33.

In the above method for producing stem cells, the blood cells or immune cells may be mononuclear cells.

In the above method for producing stem cells, the stem cells may be induced pluripotent stem (iPS) cells.

In the above method for producing stem cells, and in the induction of stem cells from the blood cells or immune cells, an induction factor RNA may be introduced into the blood cells or immune cells.

In the above method for producing stem cells, and in the induction of the stem cells from the blood cells or immune cells, an RNA virus vector may be used. The RNA virus vector may be a single-stranded RNA virus vector. The RNA virus vector may be a single-stranded positive RNA virus vector. The RNA virus vector may be a single-stranded negative RNA virus vector. The RNA virus vector may be a non-integrating RNA virus vector. The RNA virus vector may be a Mononegavirales order virus vector. The RNA virus vector may be a Paramyxoviridae family virus vector. The RNA virus vector may be a Respirovirus genus virus vector. The RNA virus vector may be a Sendai virus vector.

In the above method for producing stem cells, and in the induction of stem cells from the blood cells or immune cells, a chimeric virus comprising viral genomic RNA harboring the induction factor RNA, and an envelope encompassing the genomic RNA and derived from a virus different from that of the genomic RNA may be used.

In the above method for producing stem cells, the stem cells may have recombined γδ-TCR genes.

A method for producing blood cells or immune cells according to an embodiment of the present invention includes preparing stem cells produced by the above method for producing stem cells and inducing blood cells or immune cells from the stem cells.

In the above method for producing blood cells or immune cells, the blood cells or immune cells may be γδ T cells.

In the above method for producing blood cells or immune cells, and in the induction of the blood cells or immune cells from the stem cells, a cell mass of the stem cells may be seeded onto feeder cells.

In the above method for producing blood cells or immune cells, the feeder cells may be stromal cells.

A method for producing stem cells according to an embodiment of the present invention includes applying (E)-4-hydroxy-3-methyl-but-2-enyl pyrophosphate and interleukin to blood cells or immune cells and inducing stem cells from the blood cells or immune cells, wherein the interleukin does not include IL-2.

In the above method for producing stem cells, the interleukin may be at least one selected from the group consisting of IL-4, IL-9, IL-18, and IL-33.

In the above method for producing stem cells, the interleukin may include IL-18 and IL-33.

In the above method for producing stem cells, the interleukin may include IL-4, IL-9, IL-18, and IL-33, and IL-4 may be applied to the blood cells or immune cells after IL-9, IL-18, and IL-33.

In the above method for producing stem cells, the blood cells or immune cells may be mononuclear cells.

In the above method for producing stem cells, the stem cells may be induced pluripotent stem (iPS) cells.

In the above method for producing stem cells, and in the induction of the stem cells from the blood cells or immune cells, an induction factor RNA may be introduced into the blood cells or immune cells.

In the above method for producing stem cells, and in the induction of the stem cells from the blood cells or immune cells, an RNA virus vector may be used. The RNA virus vector may be a single-stranded RNA virus vector. The RNA virus vector may be a single-stranded positive RNA virus vector. The RNA virus vector may be a single-stranded negative RNA virus vector. The RNA virus vector may be a non-integrating RNA virus vector. The RNA virus vector may be a Mononegavirales order virus vector. The RNA virus vector may be a Paramyxoviridae family virus vector. The RNA virus vector may be a Respirovirus genus virus vector. The RNA virus vector may be a Sendai virus vector.

In the above method for producing stem cells, and in the induction of stem cells from blood cells or immune cells, a chimeric virus comprising viral genomic RNA harboring the induction factor RNA and an envelope encompassing the genomic RNA and derived from a virus different from that of the genomic RNA may be used.

In the above method for producing stem cells, the stem cells may have recombined γδ-TCR genes.

A method for producing blood cells or immune cells according to an embodiment of the present invention includes preparing stem cells produced by the above method for producing stem cells and inducing blood cells or immune cells from the stem cells.

In the above method for producing blood cells or immune cells, the blood cells or immune cells may be γδ T cells.

In the above method for producing blood cells or immune cells, and in the induction of the blood cells or immune cells from the stem cells, a cell mass of the stem cells may be seeded onto feeder cells.

In the above method for producing blood cells or immune cells, the feeder cells may be stromal cells.

A method for producing γδ T cells according to an embodiment of the present invention includes inducing differentiation of γδ T cells from pluripotent stem cells derived from T cells.

In the above method for producing γδ T cells, the pluripotent stem cells may be derived from γδ T cells.

In the above method for producing γδ T cells, the γδ T cells induced from the pluripotent stem cells may express γδ T cell receptors.

In the above method for producing γδ T cells, the pluripotent stem cells may have recombined γδ T cell receptor genes.

In the above method for producing γδ T cells, the differentiated γδ T cells may express γδ T cell receptors.

In the above method for producing γδ T cells, the differentiation of γδ T cells from pluripotent stem cells may include inducing hematopoietic stem progenitor cells from the pluripotent stem cells and inducing the γδ T cells from the hematopoietic stem progenitor cells.

In the above method for producing γδ T cells, the cells may be cultured feeder-free during the induction of hematopoietic stem progenitor cells from the pluripotent stem cells.

In the above method for producing γδ T cells, the cells may be cultured in suspension during the induction of hematopoietic stem progenitor cells from the pluripotent stem cells.

In the above method for producing γδ T cells, the hematopoietic stem progenitor cells may be induced from the pluripotent stem cells in a medium containing at least one of bone morphogenetic protein 4 (BMP-4), VEGF, and bFGF.

In the above method for producing γδ T cells, the hematopoietic stem progenitor cells may be induced from pluripotent stem cells in a medium containing an activin receptor-like kinase inhibitor.

In the above method for producing γδ T cells, the cells may be cultured in a medium containing at least one of BMP-4, VEGF, and bFGF, followed by culturing the cells in a medium containing at least one of VEGF, bFGF, SCF, TPO, and FLT3L during the induction of hematopoietic stem progenitor cells from the pluripotent stem cells.

In the above method for producing γδ T cells, the hematopoietic stem progenitor cells may be cultured on feeder cells, which may be OP9 cells.

A γδ T cell according to an embodiment of the present invention is a γδ T cell differentiated from pluripotent stem cells derived from γδ T cells.

A pharmaceutical composition for cancer treatment according to an embodiment of the present invention comprises γδ T cells differentiated from pluripotent stem cells derived from γδ T cells.

### Effects of the Invention

According to the present invention, it is possible to provide a method for efficiently producing γδ T cells.

### BRIEF DESCRIPTION OF THE DRAWINGS

FIG. 1 is a schematic diagram showing VJ gene recombination.

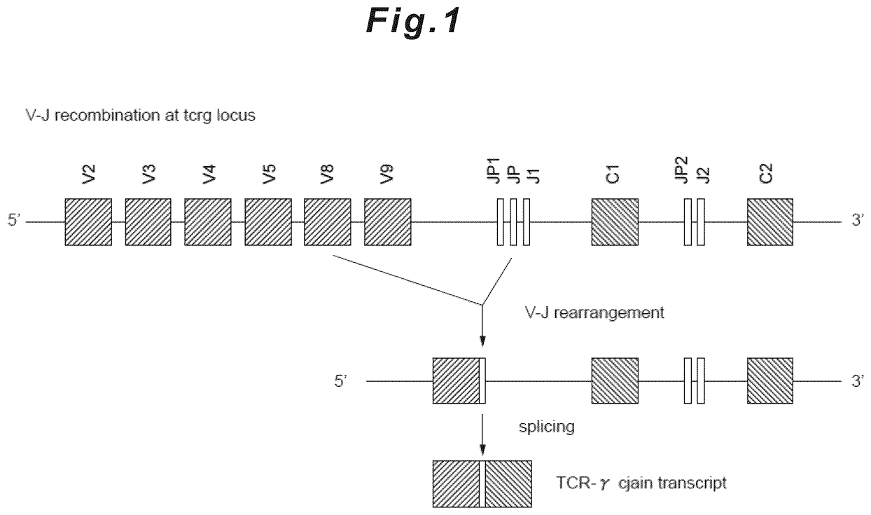

FIG. 2 is a photograph of the induced stem cells according to Example 1.

FIG. 3 is a photograph showing the results of PCR analysis of the genome of the induced stem cells according to Example 1.

FIG. 4 is a graph showing the proportion of γδ T cells according to Example 2.

FIG. 5 is a graph showing the number of γδ T cells induced from 2×10\hat{ }4 mononuclear cells according to Example 2.

FIG. 6 is a photograph of the induced stem cells according to Example 2.

FIG. 7 is a photograph of the induced stem cells according to Example 2.

FIG. 8 is a graph showing the number of colonies of induced stem cells according to Example 4.

FIG. 9 is a dot plot from a flow cytometer showing the results of Example 5.

FIG. 10 is a dot plot from a flow cytometer showing the results of Example 5.

FIG. 11 is a dot plot from a flow cytometer showing the results of Example 5.

FIG. 12 is a photograph showing the results of PCR analysis of the genome of the induced stem cells according to Example 6.

FIG. 13 is a graph showing the number of colonies of induced stem cells according to Example 7.

FIG. 14 is a photograph showing the results of PCR analysis of the genome of the induced stem cells according to Example 7.

FIG. 15 is a photograph showing the results of PCR analysis of the genome of the induced stem cells according to Example 7.

FIG. 16 is a dot plot from a flow cytometer showing the results of Example 8.

FIG. 17 is a dot plot from a flow cytometer showing the results of Example 8, Comparative Example 1, and Comparative Example 2.

FIG. 18 is a dot plot from a flow cytometer showing the results of Example 9.

FIG. 19 is a photograph of the cells according to the results of Example 9.

### DETAILED DESCRIPTION OF EMBODIMENTS

Hereinafter, embodiments of the present invention will be described in detail. The embodiments described below are provided as examples to concretize the technical idea of the present invention, and the technical idea of the present invention is not limited to the specific combination of components described hereinafter. The technical idea of the present invention may be modified in various ways within the scope of the claims.

A method for producing stem cells according to the embodiment includes applying tetrakis-pivaloyloxymethyl-2-(thiazol-2-ylamino) ethylidene-1, 1-bisphosphonate (PTA) to blood cells or immune cells, and inducing stem cells from blood cells or immune cells. The method for producing stem cells according to the embodiment may further include applying interleukin to the blood cells or immune cells. Examples of interleukins combined with PTA include IL-2, IL-4, IL-9, IL-18, and IL-33, but are not limited thereto. The interleukin may not include IL-2.

In addition, the method for producing stem cells according to the embodiment includes applying bisphosphonate or (E)-4-hydroxy-3-methyl-but-2-enyl pyrophosphate (HMBPP) and interleukin to blood cells or immune cells, and inducing stem cells from the blood cells or immune cells. The interleukin to be used in combination with bisphosphonate or HMBPP may include at least one selected from the group consisting of IL-4, IL-9, IL-18, and IL-33. The interleukin may not include IL-2.

The interleukin to be used in combination with bisphosphonate or HMBPP may, for example, include IL-18 and IL-33. The interleukin to be used in combination with bisphosphonate or HMBPP may, for example, include IL-4 and IL-18. The interleukin to be used in combination with bisphosphonate or HMBPP may, for example, include IL-9 and IL-18. The interleukin to be used in combination with bisphosphonate or HMBPP may, for example, include IL-4, IL-9, and IL-18. The interleukin to be used in combination with bisphosphonate or HMBPP may, for example, include IL-4, IL-18, and IL-33. In this case, IL-4 may be applied after IL-18 and IL-33 to the blood cells or immune cells. The interleukin to be used in combination with bisphosphonate or HMBPP may, for example, include IL-4, IL-9, IL-18, and IL-33. In this case, IL-4 may be applied after IL-9, IL-18, and IL-33 to the blood cells or immune cells.

The interleukin to be used in combination with bisphosphonate may, for example, include IL-4. The interleukin to be used in combination with bisphosphonate may, for example, include IL-4 and IL-18. The interleukin to be used in combination with bisphosphonate may, for example, include IL-4, IL-9, and IL- 18.

The interleukin to be used in combination with bisphosphonate may, for example, include IL-9. The interleukin to be used in combination with bisphosphonate may, for example, include IL-9 and IL-18.

The interleukin to be used in combination with bisphosphonate may, for example, include IL-18.

Furthermore, a method for producing stem cells according to the embodiment includes applying bisphosphonate or (E)-4-hydroxy-3-methyl-but-2-enyl pyrophosphate (HMBPP) to blood cells or immune cells, and inducing stem cells from blood cells or immune cells, wherein IL-2 is not applied to the blood cells or immune cells. Preferably, any interleukin containing IL-2 is not applied to the blood cells or immune cells.

Examples of bisphosphonates include zoledronic acid, pamidronic acid, alendronic acid, risedronic acid, ibandronic acid, incadronic acid, etidronic acid, minodronic acid, PTA, salts thereof, and hydrates thereof.

The stem cells may, for example, be induced pluripotent stem cells (iPS cells).

The blood cells or immune cells may be derived from humans or from non-human animals. The blood cells may be isolated from blood, which may include, for example, peripheral blood and cord blood, but are not limited thereto. The blood may be collected from adults or minors. During the blood collection, anticoagulants such as ethylenediaminetetraacetic acid (EDTA), heparin, or biological product standards blood preservation solution A (ACD-A) solution may be used. The immune cells may be isolated from blood, bone marrow, thymus, lymph, body fluids, and tissues. Examples of the body fluids include ascites, pleural effusion, and pericardial fluid. Examples of the tissues include tumor tissues.

The blood cells may include nucleated cells such as monocytes, neutrophils, eosinophils, basophils, and lymphocytes, but do not include erythrocytes, granulocytes, or platelets. The blood cells may also include vascular endothelial progenitor cells, hematopoietic stem and progenitor cells, T cells, or B cells. T cells may be αβ T cells or γδ T cells. The blood cells may not be γδ T cells. Examples of the immune cells include lymphocytes, which may include T cells, NK cells, or B cells.

When applying PTA or bisphosphonate to the blood cells or immune cells, PTA or bisphosphonate may be added to the culture medium in which the blood cells or immune cells are cultured. The concentration of PTA or bisphosphonate in the medium may be, for example, between 0.5 μmol/L and 50 μmol/L, between 1 μmol/L and 50 μmol/L, between 3 μmol/L and 20μmol/L, or between 5 μmol/L and 12 μmol/L. The blood cells or immune cells may be cultured in the medium with added PTA or bisphosphonate for more than 1 day, more than 2 days, or more than 3 days. The blood cells or immune cells may be cultured in the medium with added PTA or bisphosphonate for less than 14 days, less than 10 days, or less than 7 days.

When applying interleukin to the blood cells or immune cells, interleukin may be added to the culture medium in which the blood cells or immune cells are cultured. The concentration of interleukin in the medium may be, for example, between 5 ng/ml and 100 ng/ml, between 10 ng/mL and 90 ng/ml, or between 15 ng/mL and 80 ng/mL. The blood cells or immune cells may be cultured in the medium with added interleukin for more than 1 day, more than 2 days, or more than 3 days. The blood cells or immune cells may be cultured in the medium with added interleukin for less than 14 days, less than 10 days, or less than 7 days. Interleukin may be added to the medium daily, every 2 days, or every 3 days.

After applying PTA or bisphosphonate to the blood cells or immune cells, interleukin may also be applied to the blood cells or immune cells. PTA or bisphosphonate and interleukin may be applied simultaneously to the blood cells or immune cells. After applying interleukin to the blood cells or immune cells, PTA or bisphosphonate may be applied.

Examples of the media for culturing the blood cells or immune cells include RPMI 1640 medium, Minimum Essential Medium (α-MEM), Dulbecco's Modified Eagle Medium (DMEM), and F12 medium, but are not limited thereto. Prior to introducing inducing factors into the blood cells or immune cells, the blood cells or immune cells may be proliferated for 1 day, 2 days, or 3 days.

Next, inducing factors are introduced into the blood cells or immune cells that have been subjected to at least PTA or bisphosphonate to induce stem cells from the blood cells or immune cells. The term “induce” in the context of stem cell induction refers to processes such as reprogramming, initialization, transdifferentiation, and cell fate reprogramming.

The inducing factors introduced into the blood cells or immune cells may be RNA. The RNA may be mRNA. The inducing factors may include, for example, OCT3/4, SOX2, KLF4, and c-MYC. An improved version of OCT3/4, referred to as M3O, may be used as an inducing factor. Other inducing factors may include LIN28A, FOXH1, LIN28B, GLIS1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF206, FOXD3, REX1, UTF1, KLF2, KLF5, ESRRB, miR-291-3p, miR-294, miR-295, NR5A1, NR5A2, TBX3, MBD3sh, TH2A, TH2B, and P53DD. These RNA inducing factors are available from TriLink. It should be noted that gene symbols are written in uppercase to refer to human genes, but this is not intended to limit the species. For example, the uppercase format does not exclude genes from mice or rats. However, in the examples, the gene symbols are listed according to the species actually used.

A method for introducing the inducing factors into the blood cells or immune cells is not limited. For example, the inducing factors may be introduced into the blood cells or immune cells using a vector. The inducing factors may also be introduced into the blood cells or immune cells through lipofection.

As the vector, an RNA virus can be used. The RNA virus may be a single-stranded RNA virus. The RNA virus may be a single-stranded positive RNA virus. The RNA virus may be a single-stranded negative RNA virus. The RNA virus may be a non-integrating RNA virus. The RNA virus may belong to the order Mononegavirales. The RNA virus may be from the Paramyxoviridae family. The RNA virus may belong to the genus Respirovirus. The RNA virus may be Sendai virus (SeV). Sendai virus belongs to the order Mononegavirales and the family Paramyxoviridae, and it contains an RNA genome and a lipid bilayer envelope that encapsulates the RNA.

As Sendai virus harboring the RNA of the inducing factors, CytoTune® (Invitrogen) may be used. The Sendai virus vector may be Sendai virus vector with an improved infection persistence. Alternatively, a stealth RNA vector may be used as the RNA virus vector. Stealth RNA vectors, such as SRV iPSC-1 Vector, SRV iPSC-2 Vector, SRV iPSC-3 Vector, and SRV iPSC-4 Vector (registered trademarks of Tokiwa Bio Corporation), may be used. Detailed descriptions of stealth RNA vectors are provided in Japanese Patent No. 4478788,Japanese Patent No. 4936482, Japanese Patent No. 5633075, and Japanese Patent No. 5963309.

The multiplicity of infection (MOI) is used as an indicator of the titer of Sendai virus. The MOI of Sendai virus may range from 0.1 to 100.0, or from 1.0 to 50.0.

The inducing factor may be introduced into the blood cells or immune cells using a chimeric virus that comprises genome RNA derived from the virus and harboring the inducing factor RNA and an envelope encompassing the genomic RNA and derived from a virus different from that of the genome RNA.

The genome RNA of the chimeric virus may be derived from Paramyxoviridae. The genome RNA of the chimeric virus may be a stealth RNA vector.

The genome RNA of the chimeric virus may include viral genes in which each function of M gene, F gene, and HN gene are entirely defected and L gene has a mutation that allows sustained gene expression among viral genes containing a nucleocapsid protein (NP) gene, a phosphoprotein (P) gene/C protein (C) gene, a matrix protein (M) gene, a fusion protein (F) gene, a hemagglutinin-neuraminidase (HN) gene and a large protein (L) gene, and the inducing factor RNA.

The NP gene, P gene/C gene, and L gene are involved in the transcription and replication of the virus vector. The F gene, M gene, and HN gene are involved in particle formation of the virus. Thus, a virus vector in which all functions of the F gene, M gene, and HN gene are defected cannot form new viral particles after infecting the cell. In the genome RNA, the F gene, M gene, and HN gene may be deleted to entirely remove the functions of the F gene, M gene, and HN gene.

The mutated L gene, which allows for sustained gene expression, encodes a large protein where the 1618th amino acid is valine. A virus vector with this mutated L gene has reduced interferon-inducing ability, making it non-cytotoxic and capable of persistent infection. Therefore, expression of the inducing factor RNA is sustained within the cell.

After the stem cells are induced from the blood cells or immune cells, siRNA targeting at least one of the NP gene, P gene, or mutated L gene may be introduced into the cells to remove the virus vector. For example, siRNA targeting the region containing the 527th or 1913th nucleotide of the mutated L gene may be introduced into the cells.

Alternatively, a target sequence for an undifferentiated cell-specific microRNA may be added to the non-coding regions of at least one of the NP gene, P gene, or mutated L gene. An example of such the undifferentiated cell-specific microRNA is miR-302a. When the stem cells are induced from the blood cells or immune cells, expression of the undifferentiated cell-specific microRNA, such as miR-302a, is induced in the cells. When the undifferentiated cell-specific microRNA binds to the target sequence, expression of at least one of the NP gene, P gene, or mutated L gene is suppressed, thereby removing the virus vector from the cells. For example, the target sequence of miR-302a may be added to the mutated L gene.

The genome RNA of the chimeric virus may comprise, from the 3′ end, the NP gene, P gene, C gene, and a mutated L gene that enables sustained gene expression. The genome RNA may include the RNA of the inducing factors between the C gene and the mutated L gene. The genome RNA may also include the RNA of a fluorescent protein between the C gene and the mutated L gene. Examples of the fluorescent proteins include Enhanced Green Fluorescent Protein (EGFP). The genome RNA may further include the RNA of a drug resistance gene between the C gene and the mutated L gene.

The envelope of the chimeric virus may be derived from, for example, the measles virus.

A chimeric virus comprising genome RNA having, from the 3′ end, the NP gene, P gene, C gene, and mutated L gene, as well as RNA for EGFP and the inducing factors between the C gene and the mutated L gene, along with an envelope encompassing the genome RNA and derived from the measles virus, was produced by requesting Tokiwa Bio to produce it as MSRV-1. A chimeric virus in which the target sequence of miR-302a is added to the mutated L gene of MSRV-1 was also produced by requesting Tokiwa Bio to produce as MSRV-2.

The multiplicity of infection (MOI) is used as an indicator of the titer of the chimeric virus. The MOI of the chimeric virus may be, for example, between 0.1 and 100.0, or between 1.0 and 50.0.

The inducing factors may be introduced into the blood cells or immune cells that are cultured under adherent conditions, or into the blood cells or immune cells that are cultured in suspension in gel media.

The blood cells or immune cells into which the inducing factors are introduced may be cultured feeder-free using a basement membrane matrix, such as Matrigel (Corning), CELLstart® (ThermoFisher), or Laminin 511 (iMatrix-511 , Nippi).

As for the medium in which the blood cells or immune cells into which the inducing factors are introduced are cultured, stem cell media such as Stemfit (Ajinomoto) and human ES/iPS media may be used.

However, the stem cell media are not limited thereto, and various stem cell media can be used. For example, Primate ES Cell Medium, mTeSR1, and TeSR2 (STEMCELL Technologies) may also be utilized. The stem cell media can be placed in dishes, wells, tubes, or the like.

The gel medium may not require stirring. Additionally, the gel medium may not contain feeder cells.

The gel medium may contain at least one substance selected from the group consisting of cadherin, laminin, fibronectin, and vitronectin.

After the introduction of inducing factors into the blood cells or immune cells, the cells may be initialized in either a liquid media that is not a gel media or a gel media.

After the introduction of inducing factors into the blood cells or immune cells and subsequent culturing, the cells into which the inducing factors were introduced may be harvested, and at least part of the harvested mixed cells may be subcultured at least once by seeding them into media. During subculturing, the clones of cells into which the inducing factors were introduced may be mixed. Alternatively, clones of different cells into which the inducing factors were introduced may be mixed. The cells into which the inducing factors were introduced may then be harvested again, and at least part of the harvested mixed cells may be seeded into media and subcultured multiple times until stem cells are established. All of the harvested mixed cells may be seeded into the media.

Here, subculturing after the cells into which the inducing factors were introduced have been harvested and at least part of the harvested mixed cells have been seeded into media refers to subculturing without distinguishing the cells based on gene expression status. For example, at the time of subculturing, the cells into which the inducing factors were introduced may be seeded into the same culture vessel without distinguishing them by gene expression status. Alternatively, subculturing after harvesting the cells and seeding at least part of the harvested mixed cells into media refers to subculturing without distinguishing cells by the degree of reprogramming. For example, at the time of subculturing, the cells into which the inducing factors were introduced may be seeded into the same culture vessel without distinguishing them by the degree of reprogramming.

Alternatively, subculturing after harvesting the cells and seeding at least part of the harvested mixed cells into media refers to subculturing without distinguishing the cells by morphology. For example, during subculturing, the cells into which the inducing factors were introduced may be seeded into the same culture vessel without distinguishing them by morphology. Alternatively, subculturing after harvesting the cells and seeding at least part of the harvested mixed cells into media refers to subculturing without distinguishing the cells by size. For example, during subculturing, the cells into which the inducing factors were introduced may be seeded into the same culture vessel without distinguishing them by size.

Alternatively, subculturing after harvesting the cells and seeding at least part of the harvested mixed cells into media refers to subculturing without cloning the cells into which the inducing factors were introduced. For example, in the case of subculturing without cloning, it is not necessary to pick up colonies formed by the cells into which the inducing factors were introduced. Additionally, it is not necessary to separate multiple colonies formed by the cells into which the inducing factors were introduced during subculturing. For example, during subculturing, the cells forming multiple different colonies may be mixed and seeded into the same culture vessel. In the case of subculturing without cloning, it is not necessary to clone single colonies formed by the cells into which the inducing factors were introduced. For example, during subculturing, colonies may be mixed and seeded into the same culture vessel.

For example, when the cells into which the inducing factors were introduced are cultured under adherent conditions, the adherent cells may be harvested, and at least part of the harvested mixed cells may be seeded into media and subcultured. For example, during subculturing, the cells may be detached from the culture vessel and at least part of the detached mixed cells may be seeded into the same culture vessel. For example, the cells may be detached from the culture vessel using a dissociation solution, and the entire population of detached mixed cells may be subcultured. Cells that have not formed colonies may also be subcultured. If the cells into which the inducing factors were introduced are cultured in suspension, the entire population of suspended cells may be subcultured.

The cells into which the inducing factors were introduced may be cultured and subcultured in a closed culture vessel. The closed culture vessel, for example, does not exchange gases, viruses, microorganisms, or impurities with the external environment. Furthermore, the cells into which the inducing factors were introduced may be expanded through two-dimensional or three-dimensional culture.

After the induced cells have been reprogrammed into stem cells and the stem cells have been established, the entirety of the adherent-cultured cells may be cryopreserved as stem cells.

For example, all the cells detached from the culture vessel using a dissociation solution may be cryopreserved as stem cells. Additionally, after the induced cells have been reprogrammed into stem cells, the entirety of the suspension-cultured cells may be cryopreserved as stem cells.

The induced stem cells may express undifferentiated cell markers such as Nanog, OCT4, and SOX2. The induced stem cells may express TERT. The induced stem cells may exhibit telomerase activity.

The induction of stem cells from blood cells or immune cells may be confirmed, for example, by observing the cell morphology. The induced stem cells may form flat colonies similar to embryonic stem cells and express alkaline phosphatase. Alternatively, the induction of stem cells from blood cells or immune cells may be confirmed by analyzing the expression of at least one undifferentiated surface marker selected from TRA-1-60, TRA-1-81, SSEA-3, SSEA-4, and SSEA-5 using a flow cytometer. TRA-1-60 is an antigen specific to iPS/ES cells and is not detected in somatic cells. Since iPS cells are generated only from TRA-1-60-positive fractions, TRA-1-60-positive cells are considered to be iPS cells.

The induced stem cells may, for example, possess a recombined γδ-TCR gene. A recombined γδ-TCR gene refers to a gene coding for a TCR in which the TCRγ region and TCRδ region have undergone recombination. The TCRγ region includes Vγ-Jγ, and the TCRO region includes Vδ-Dδ-Jδ. The induced stem cells may possess a recombined γδ-TCR gene that includes, for example, the J1/J2 gene.

A method for producing blood cells or immune cells according to the present embodiment includes preparing the stem cells produced by the method described above and inducing blood cells or immune cells from the stem cells.

A method for inducing the blood cells or immune cells from the stem cells is not particularly limited. For example, the prepared cells may be cultured for four days in a medium containing a glycogen synthase kinase-3 (GSK3) inhibitor, such as CHIR99021, bone morphogenetic protein-4 (BMP-4), and growth factors such as vascular endothelial growth factor (VEGF). Furthermore, the cells may be cultured for two days in a medium containing an ALK5 inhibitor, such as SB431542, growth factors such as VEGF and basic fibroblast growth factor (bFGF), and stem cell factor (SCF). Furthermore, the cells may be cultured for two days in a medium containing growth factors such as VEGF, SCF, interleukins such as IL-3 and IL-6, cytokines such as Flt3L, and erythropoietin (EPO). The cells may then be cultured in a medium containing SCF, interleukins such as IL-6, and EPO. In this way, blood cells or immune cells are induced.

Alternatively, the stem cells may be seeded onto stromal cells, and the blood cells or immune cells may be induced from the stem cells. The stromal cells may be bone marrow-derived. The stromal cells may be OP9 cells. OP9 cells do not produce macrophage colony-stimulating factor (M-CSF) and have the function of supporting the differentiation of stem cells into blood cells or immune cells. For example, the stem cell colonies may be divided into multiple cell aggregates, and the stem cell aggregates may be seeded onto OP9 cells as feeder cells. In this way, the blood cells or immune cells are induced from the stem cells. The induced blood cells or immune cells are, for example, CD34-positive and CD43-positive.

The induced blood cells or immune cells may be γδ-type T cells.

A method for producing γδ T cells according to the present embodiment includes differentiating γδ T cells from pluripotent stem cells induced from T cells. The pluripotent stem cells are, for example, iPS cells. The pluripotent stem cells may be induced from γδ T cells. The pluripotent stem cells may be induced from γδ T cells expressing a γδ T cell receptor.

A method for inducing the pluripotent stem cells from T cells is not particularly limited. For example, the pluripotent stem cells may be induced from T cells using the same method as the method for producing the stem cells described above. Alternatively, the pluripotent stem cells may be induced from T cells stimulated with IL-2 and zoledronic acid. The induced pluripotent stem cells may have recombined γδ TCR genes.

The differentiation of γδ T cells from the pluripotent stem cells may include inducing hematopoietic stem progenitor cells from the pluripotent stem cells and inducing γδ T cells from the hematopoietic stem progenitor cells. During the induction of hematopoietic stem progenitor cells from the pluripotent stem cells, the cells may be cultured feeder-free. During the induction of hematopoietic stem progenitor cells from the pluripotent stem cells, the cells may be cultured in suspension.

The hematopoietic stem progenitor cells may be induced from the pluripotent stem cells in a medium containing at least one of BMP-4, VEGF, and bFGF. The hematopoietic stem progenitor cells may be induced from the pluripotent stem cells in a medium containing BMP-4, VEGF, and bFGF.

The hematopoietic stem progenitor cells may be induced from the pluripotent stem cells in a medium containing an activin receptor-like kinase inhibitor. The activin receptor-like kinase inhibitor may be added to a medium containing BMP-4, VEGF, and bFGF.

During the induction of the hematopoietic stem progenitor cells from the pluripotent stem cells, the cells may be cultured in a medium containing at least one of BMP-4, VEGF, and bFGF, and subsequently cultured in a medium containing at least one of VEGF, bFGF, SCF, TPO, and FLT3L.

During the induction of the hematopoietic stem progenitor cells from the pluripotent stem cells, the cells may be cultured in a medium containing BMP-4, VEGF, and bFGF, and subsequently cultured in a medium containing VEGF, bFGF, and SCF.

During the induction of the hematopoietic stem progenitor cells from the pluripotent stem cells, the cells may be cultured in a medium containing BMP-4, VEGF, and bFGF, and subsequently cultured in a medium containing VEGF, bFGF, SCF, TPO, and FLT3L.

The hematopoietic stem progenitor cells may be cultured on feeder cells. The feeder cells may be OP9 cells.

The γδ T cells differentiated from the pluripotent stem cells may express a γδ T cell receptor. The γδ T cells may be used as a pharmaceutical composition for cancer treatment.

### Example 1

A PTA-containing medium according to Example 1 was prepared using RPMI 1640 medium (Gibco) containing 1 μmol/L to μmol/L of tetrakis-pivaloyloxymethyl-2-(thiazol-2-ylamino) ethylidene-1, 1-bisphosphonate (PTA, Techno Suzuta), 10% fetal bovine serum (Life Technologies), 1.0×10\hat{ }−5 mol/L 2-mercaptoethanol (Nacalai Tesque), 100 U/mL penicillin, and 100 μg/mL streptomycin (Life Technologies).

Human peripheral blood mononuclear cells were added to the PTA-containing medium according to Example 1, and a medium containing approximately 1×10\hat{ }6 mononuclear cells was placed into a 24-well plate (Day 1). To the medium, 1 μL of 20 μg/mL IL-2 was added daily. On Day 3, the medium containing cells was collected from the plate, centrifuged, and the supernatant was removed. Then, 2 mL of fresh PTA-containing medium according to Example 1 was added to the cells, and 1 mL of the medium was placed into two wells of a 24-well plate.

On Day 6, the medium containing cells was collected from the plate, centrifuged, and the supernatant was removed. Then, the PTA-containing medium according to Example 1 was added to the cells, and a medium containing 2×10\hat{ }4 mononuclear cells was placed into a 96-well plate. KLF4, OCT3/4, SOX2, and c-MYC were introduced into the cells using MSRV (Tokiwa Bio). The multiplicity of infection (MOI) was adjusted to 5.

On Day 7, fresh PTA-containing medium according to Example 1 was added to the medium containing the cells collected from the 96-well plate, and the cells were placed into a 6-well plate coated with laminin (iMatrix-511, Nippi). On Days 8, 10, and 12, stem cell medium (Stem Fit, Ajinomoto) was added, and subsequently, the medium was replaced with fresh stem cell medium.

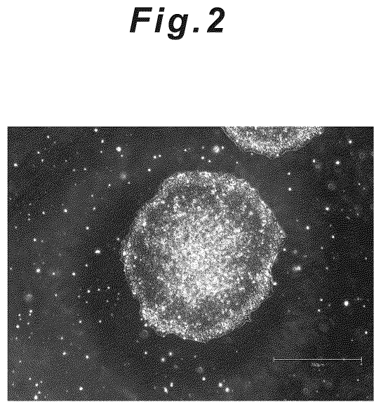

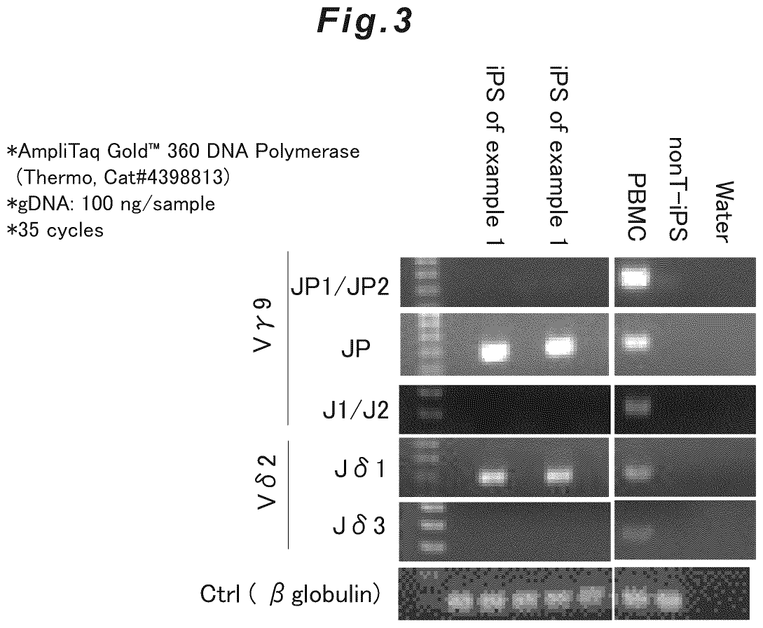

On Day 14, multiple colonies of iPS cells were confirmed. A photograph of the established iPS cells is shown in FIG. 2. Additionally, genomic DNA was extracted from the iPS cells, and the presence of the recombined Vγ9 gene was analyzed by PCR and electrophoresis. Genomic DNA from mononuclear cells (PBMC) was used as a positive control, and genomic DNA from iPS cells lacking the recombined Vγ9 gene was used as a negative control. As shown in FIG. 3, the iPS cells established in Example 1 were confirmed to possess a recombined Vγ9 gene with a JP gene and a recombined V82 gene with a Jδl gene.

### Example 2

A zoledronic acid-containing medium according to Example 2 was prepared using RPMI 1640 medium (Gibco) containing 5 μmol/L of zoledronic acid (Zol, Sigma-Aldrich), 10% fetal bovine serum (Life Technologies), 1.0×10\hat{ }−5 mol/L 2-mercaptoethanol (Nacalai Tesque), 100 U/mL penicillin, and 100 μg/mL streptomycin (Life Technologies).

Human peripheral blood mononuclear cells were added to the zoledronic acid-containing medium according to Example 2, and a medium containing approximately 1×10\hat{ }6 mononuclear cells was placed into a 24-well plate (Day 1). To the medium of the first group of mononuclear cells, 1 μL of 20 μg/mL IL-2 was added daily. To the medium of the second group, 1 μL of 20 μg/mL IL-9 was added daily. To the medium of the third group, 1 μL of 20 μg/mL IL-33 was added daily. To the medium of the fourth group, 1 μL each of 50 μg/mL IL-18 and 500 μg/mL IL-33 was added daily. To the medium of the fifth group, 1 μL each of 5 g/mL IL-4, 50 μg/mL IL-18, and 500 μg/mL IL-33 was added daily. To the medium of the sixth group, 1 μL each of 5 μg/mL IL-4, 2 μg/mL IL-9, 50 μg/mL IL-18, and 500 μg/mL IL-33 was added daily.

On Day 3, the medium containing the cells was collected from the plate, centrifuged, and the supernatant was removed. Then, 2 mL of fresh zoledronic acid-containing medium according to Example 2 was added to the cells, and 1 mL of the medium was placed into two wells of a 24-well plate.

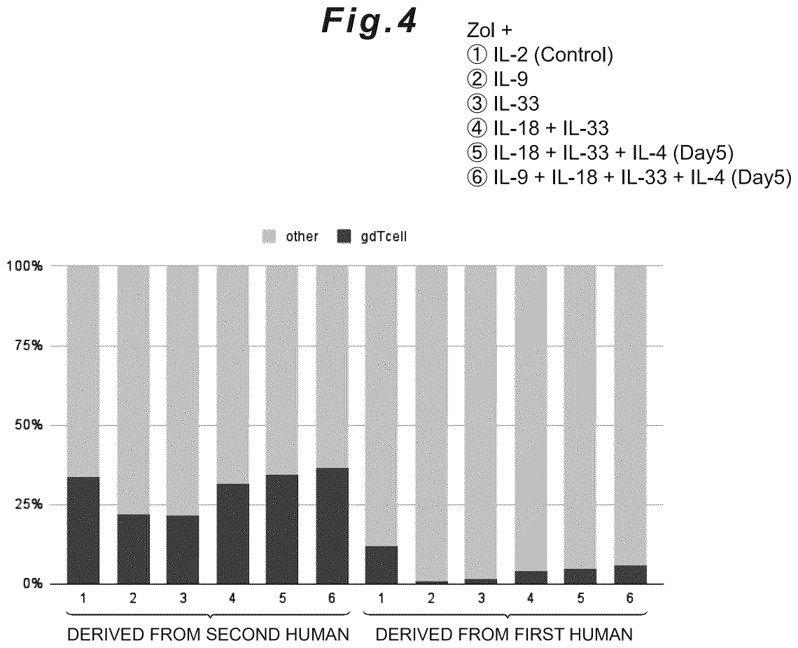

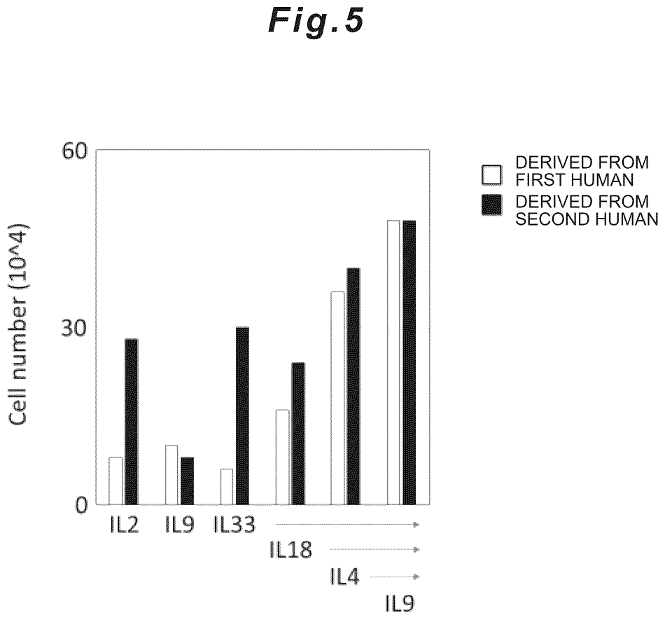

The ratio of γδ T cells analyzed by flow cytometry using anti-δ9 and anti-δ2 antibodies on Day 6 is shown in FIG. 4. The number of γδ T cells induced from 2×10\hat{ }4 mononuclear cells is shown in FIG. 5.

On Day 6, the medium containing cells was collected from the plate, centrifuged, and the supernatant was removed. Then, the zoledronic acid-containing medium according to Example 2 was added to the cells, and a medium containing 2×10\hat{ }4 mononuclear cells was placed into a 96-well plate. KLF4 , OCT3/4, SOX2, and c-MYC were introduced into the cells using MSRV (Tokiwa Bio). The MOI was adjusted to 5.

On Day 7, fresh zoledronic acid-containing medium according to Example 2 was added to the medium containing the cells collected from the 96-well plate, and the cells were placed into a 6-well plate. On Days 8, 10, and 12, stem cell medium (Stem Fit, Ajinomoto) was added, and subsequently, the medium was replaced with fresh stem cell medium.

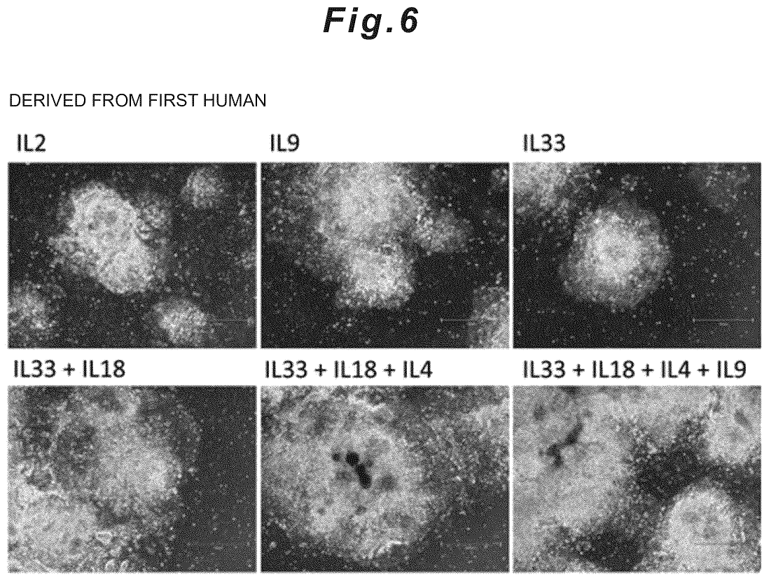

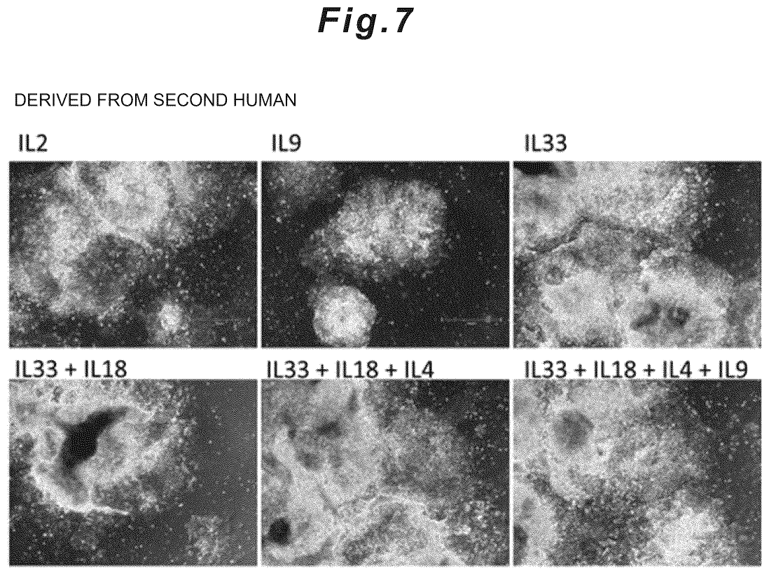

On Day 14, multiple colonies of iPS cells were confirmed. Photographs of the established iPS cells are shown in FIGS. 6 and 7.

### Example 3

iPS cells were induced from mononuclear cells in the same manner as in Example 2, except that the zoledronic acid in the zoledronic acid-containing medium according to Example 2 was replaced with HMBPP. It should be noted that IL-2 was used only as an interleukin.

### Example 4

Human peripheral blood mononuclear cells were added to the zoledronic acid-containing medium according to Example 2, and a medium containing approximately 1×10\hat{ }6 mononuclear cells was placed into a 24-well plate (Day 1). No interleukin was added to the medium of the first group. To the medium of the second group, 1 μL of 20 μg/mL IL-2 was added daily. To the medium of the third group, 1 μL of 20 μg/mL IL-9 was added daily. To the medium of the fourth group, 1 μL of 20 μg/mL IL-18 was added daily. To the medium of the fifth group, 1 μL of 20 μg/mL IL-4 was added daily. To the medium of the sixth group, 1 μL each of 20 μg/mL IL-9 and 20 μg/mL IL-18 was added daily. To the medium of the seventh group, 1 μL each of 20μg/mL IL-4 and 20 μg/mL IL-18 was added daily. To the medium of the eighth group, 1 L each of 20 μg/mL IL-4, 20 μg/mL IL-9, and 20 μg/mL IL-18 was added daily. In addition, a zoledronic acid-free medium identical to the zoledronic acid-containing medium according to Example 2, except for the absence of zoledronic acid, was prepared, and 1 μL of 20 μg/mL IL-2 was added daily to the zoledronic acid-free medium of the ninth group of mononuclear cells.

On the third day, the culture medium containing the cells was collected from the plate, centrifuged, and the supernatant was removed. Thereafter, 2 mL of the zoledronic acid-containing medium according to Example 2 was added to the first to eighth cell groups, and 1 mL each was dispensed into two wells of a 24-well plate. Additionally, 2 mL of a new zoledronic acid-free medium was added to the ninth cell group, and 1 mL each was dispensed into two wells of a 24-well plate.

On day 6, the medium containing the cells was collected from the plate, centrifuged, and the supernatant was removed. Fresh zoledronic acid-containing medium according to Example 2 was added to the cell groups from 1 to 8, and zoledronic acid-free medium was added to the cell group 9. The medium containing 2×10\hat{ }4 mononuclear cells was placed into a 96-well plate. Using MSRV (Tokiwa Bio), KLF4, OCT3/4, SOX2, and c-MYC were introduced into the cells. The multiplicity of infection (MOI) was adjusted to 5.

On day 7, fresh zoledronic acid-containing medium according to Example 2 was added to the medium containing the cell groups 1 to 8 collected from the 96-well plate, and fresh zoledronic acid-free medium was added to the medium containing cell group 9. The cells were transferred to a 6-well plate. On days 8, 10, and 12, stem cell medium (Stem Fit, Ajinomoto) was added, and subsequently, the medium was replaced using the same stem cell medium.

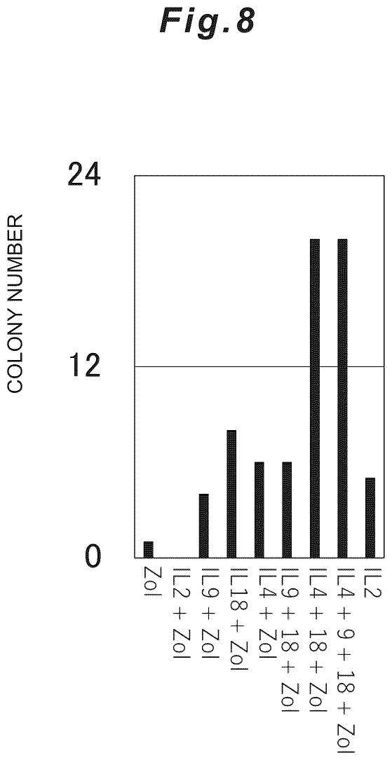

On day 14, multiple colonies of iPS cells were confirmed. The number of colonies formed is shown in FIG. 8.

### Example 5

αMEM medium supplemented with a final concentration of 20% FBS and 1% penicillin/streptomycin was prepared as an OP9 cell culture medium.

OP9 cell culture medium supplemented with a final concentration of 100 mmol/L vitamin C, 10 ng/ML IL-7, 10 μg/mL FLT3 ligand, and 10 μg/mL stem cell factor (SCF) was prepared as a differentiation induction medium.

Colonies of iPS cells induced using IL-2 as the only interleukin in Examples 2 and 3 were scraped from the dish using a cell scraper and divided into multiple cell clusters by pipetting. A dish on which OP9 cells were cultured was prepared as the feeder cells. The OP9 cells were cultured in the OP9 medium. Multiple cell clusters of iPS cells were seeded onto the feeder cells, and stem cell medium containing 10 μmol/L Y27632 was added. The cells were cultured for two days. On the second day, the medium was replaced with the OP9 medium supplemented with vitamin C, and the cells were cultured for an additional 12 days. On days 5, 9, and 13, half of the medium was removed from the dish and replaced with fresh OP9 medium supplemented with vitamin C.

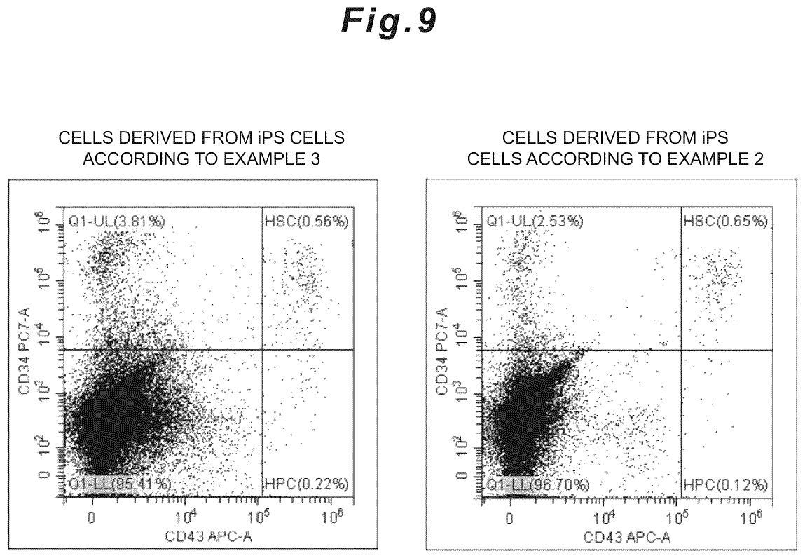

During the 14-day culture, the differentiation of iPS cells into hematopoietic progenitor cells was observed. The results of flow cytometry analysis of the cells on day 14 are shown in FIG. 9. The cells were confirmed to be positive for CD34 and CD43, which are markers of hematopoietic cells. The hematopoietic progenitor cells obtained on day 14 were seeded onto OP9/DLL1 cells and cultured for an additional 25 days. During this time, the differentiated cells were transferred onto new OP9/DLL1 cells every 4 or 5 days.

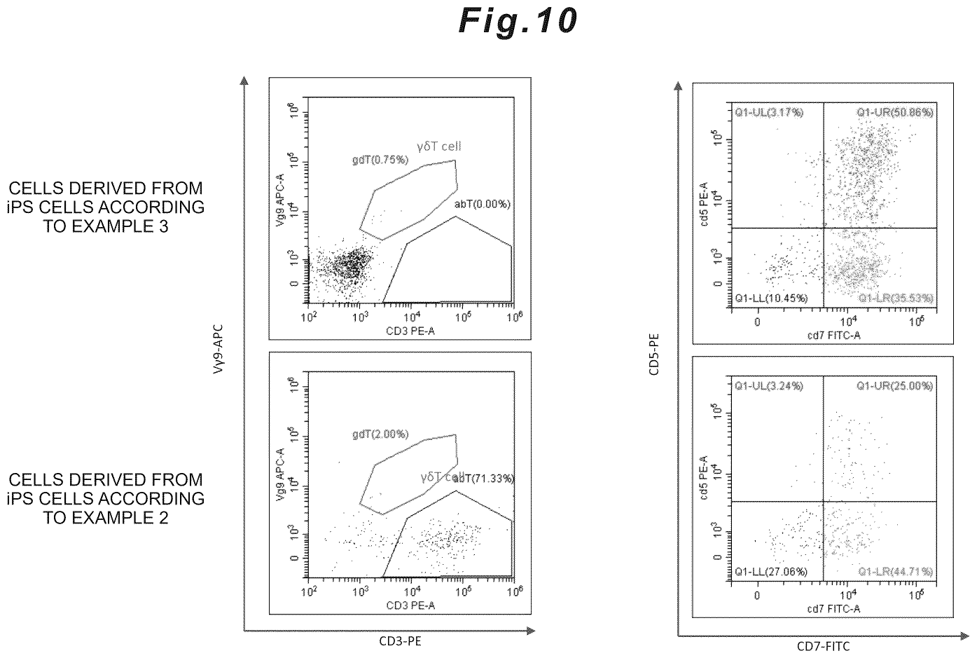

The results of flow cytometry analysis of the cells cultured on OP9/DLL1 for 28 days are shown in FIG. 10. As shown in FIG. 10, the cells were positive for both CD5-PE and CD7-FITC, which are markers of lymphocyte progenitor cells. Additionally, as shown in the left graph of FIG. 10, a population of cells positive for γδ TCR-APC, a marker of γδ T cells, was identified within the population positive for the Pan-T cell marker CD3-PE. At the same time, a population of αβ T cells positive only for CD3-PE was also identified.

To induce the cells that is induced T cell progenitors into γδ T cells, the cells were strongly stimulated through the γδ T cell receptor (TCR). As stimulants, HMBPP (0.01 μg/mL to 1 μg/mL) or zoledronic acid (5 μmol/L) was added to the culture medium, and the cells were cultured up to day 35. In some culture samples, feeder cells expressing Notch ligand were replaced with OP9 cells that did not express the Notch ligand to inhibit differentiation into αβ T cells.

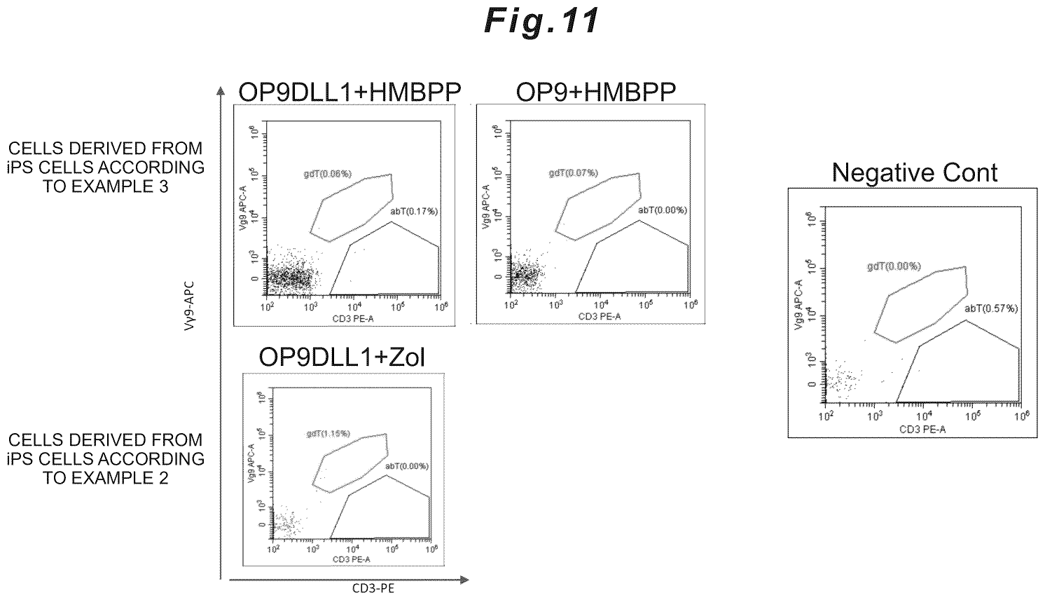

The results of flow cytometry analysis of the cells on day 35 are shown in FIG. 11. It was confirmed that CD3 and γδ TCR (Vγ9) were expressed on the surface of the cells. The expression of these markers was higher when the feeder cells were OP9-DLL1 cells compared to OP9 cells. In the negative control, where no stimulants were added, no marker expression was detected.

### Example 6

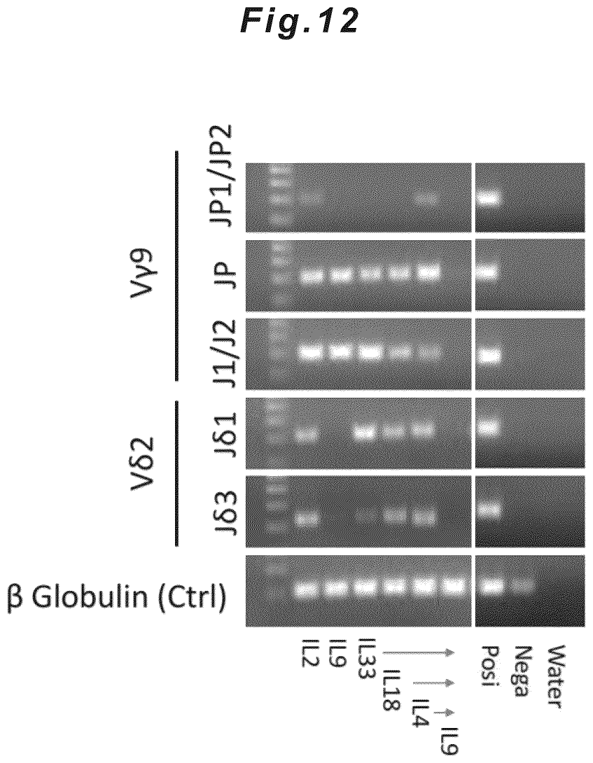

Genomic DNA was extracted from the iPS cells established in Example 2, and the presence of the recombined Vγ9 gene was analyzed by PCR and electrophoresis. Genomic DNA from mononuclear cells (PBMC) was used as a positive control, and genomic DNA from iPS cells lacking the recombined Vγ9 gene was used as a negative control. As shown in FIG. 12, the iPS cells established in Example 1 were confirmed to possess a recombined Vγ9 gene with the JP gene and a recombined Vδ2 gene with the Jδ1 gene.

### Example 7

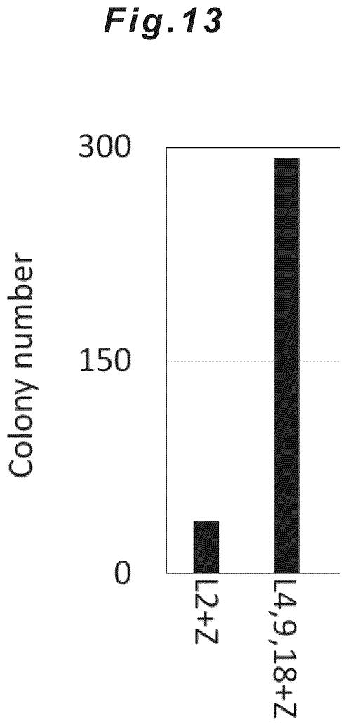

iPS cells were established in the same manner as Example 4, using IL-2 as the only interleukin added to the zoledronic acid-containing medium. Additionally, iPS cells were established in the same manner as Example 4, using a combination of IL-4, IL-9, and IL-18 added to the zoledronic acid-containing medium. As shown in FIG. 13, the number of colonies of iPS cells established was higher when IL-4, IL-9, and IL-18 were added to the medium compared to when only IL-2 was added.

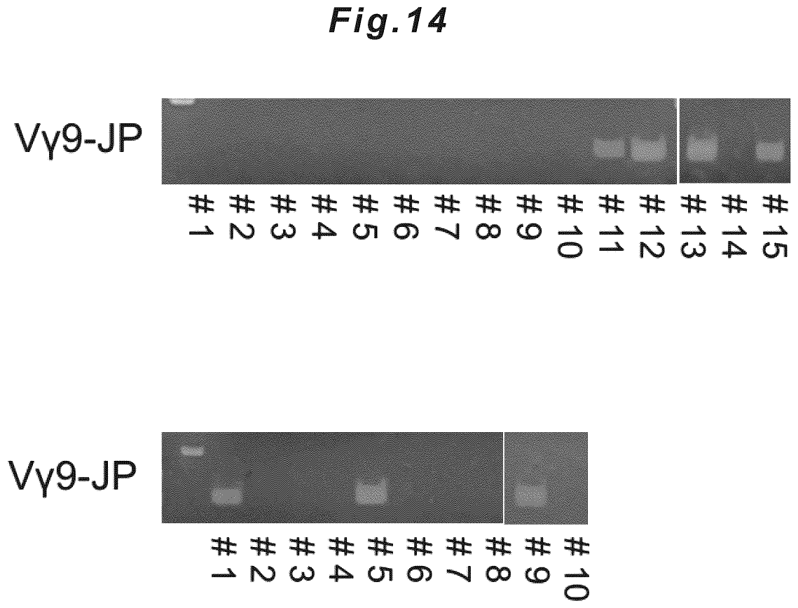

In the iPS cell lines established using only IL-2, as shown in FIG. 14, 7 out of 27 iPS cell lines possessed a recombined Vγ9 gene with the JP gene. Thus, 28% of the iPS cell lines established using only IL-2 contained the recombined Vγ9 gene with the JP gene.

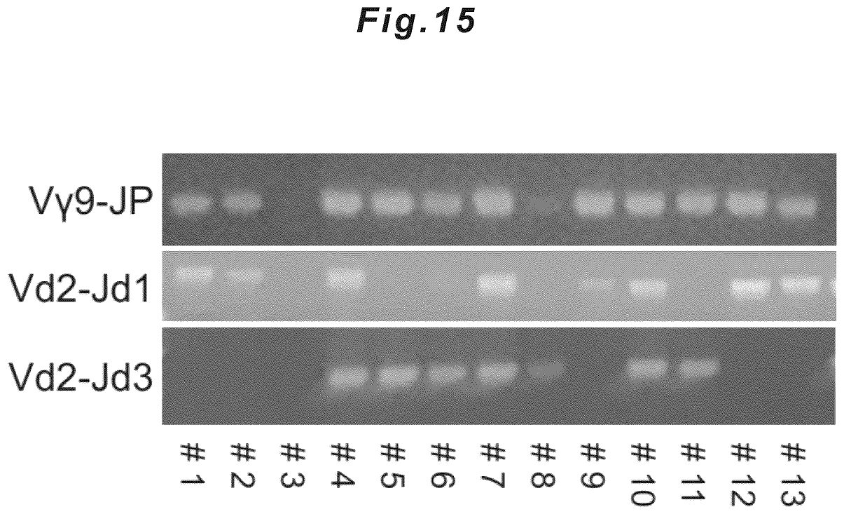

In the iPS cell lines established using IL-4, IL-9, and IL-18, as shown in FIG. 15, 12 out of 13 iPS cell lines possessed a recombined Vγ9 gene with the JP gene. Thus, 92% of the iPS cell lines established using IL-4, IL-9, and IL-18 contained the recombined Vγ9 gene with the JP gene.

### Example 8

γδ T-iPS cells induced from γδ T cells in medium supplemented with IL-2 and zoledronic acid were prepared, and the iPS cells were cultured in adherent conditions. The iPS cells were collected using TrypLE Select and resuspended in iPS cell medium (Puel, I Peace, Inc.). The iPS cells were placed in a low-attachment well plate, and 10 μmol/L ROCK inhibitor and 10 μmol/L GSK-3 inhibitor were added to the medium. The cells were cultured at 37° C. in a 5% CO2 environment. The cell suspension was then collected, centrifuged, and the supernatant was removed.

A serum-free medium (Gibco StemPro-34 SFM) supplemented with a substitute for L-glutamine (Gibco GlutaMAX supplement), 50 μg/mL ascorbic acid 2-phosphate, 40 mmol/L monothioglycerol (MTG), insulin-transferrin-selenium solution (ITS), and penicillin/streptomycin was prepared as the differentiation medium.

Cells recovered from the cell suspension were resuspended in the differentiation medium, which was supplemented with 50 ng/mL bone morphogenetic protein 4 (BMP-4), 50 ng/mL VEGF, and 50 ng/mL bFGF. The cells were cultured at 37° C. in a 5% CO2 environment. After 24 hours, 6 μmol/L of activin receptor-like kinase inhibitor (SB431542) was added to the medium.

On day 4 of culture in the differentiation medium, the cell suspension was collected, centrifuged, and the supernatant was removed. The cells were resuspended in differentiation medium supplemented with 50 ng/mL VEGF, 50 ng/mL bFGF, and 50 ng/ml SCF, and cultured at 37° C. in a 5% CO2 environment.

On day 7 of culture in the differentiation medium, the cell suspension was collected, centrifuged, and the supernatant was removed. The cells were resuspended in differentiation medium supplemented with 50 ng/mL VEGF, 50 ng/mL bFGF, 50 ng/mL SCF, 30 ng/mL TPO, and 10 ng/mL FLT3L, and cultured at 37° C. in a 5% CO2 environment. This process was repeated every 2 to 3 days until day 14 to induce the formation of embryoid bodies.

On day 14 of culture in the differentiation medium, the cell suspension was collected, and the embryoid bodies were disrupted by pipetting. The disrupted embryoid bodies were passed through a 40-μm filter to isolate hematopoietic progenitor cells.

OP9/DLL1 cells were prepared as feeder cells. Additionally, αMEM medium supplemented with FBS, GlutaMAX supplement, and penicillin/streptomycin was prepared as the OP9 medium. The OP9/DLL1 cells were seeded into a 6-cm dish and cultured overnight in OP9 medium.

The hematopoietic progenitor cells were resuspended in OP9 medium supplemented with SCF, IL-7, FLT3L, and ascorbic acid 2-phosphate and seeded onto the OP9/DLL1 cells. The medium was replaced with fresh OP9 medium every 2 to 3 days, and the cells were passaged onto new OP9/DLL1 cells every week, continuing for 3 weeks.

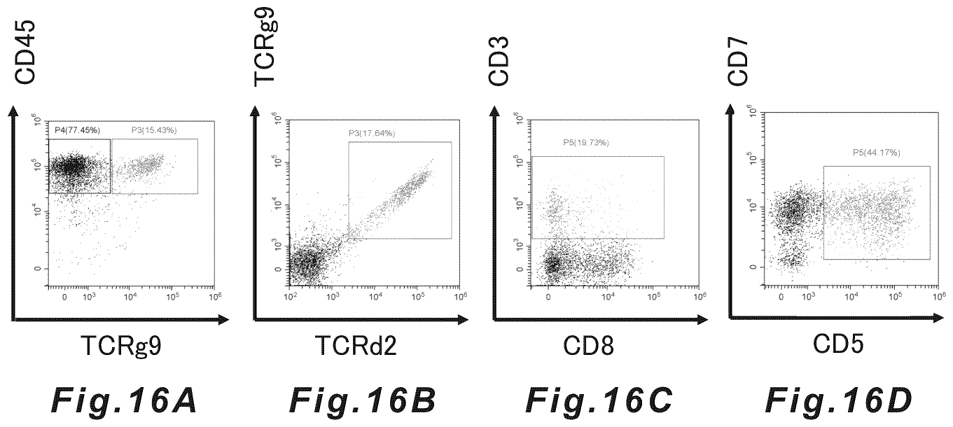

Flow cytometry analysis of the cells cultured for 3 weeks revealed that cells positive for CD45, CD3, and TCRγ9, as well as cells positive for CD45 and TCRγ9, and cells positive for both TCRγ9 and TCR82, were detected, indicating the induction of γδ T cells (see FIG. 16). Additionally, cells positive for CD45 and negative for TCRγ9, cells positive for CD3 and negative for CD8, cells positive for CD3 and CD8, cells positive for CD7 and negative for CD5, and cells positive for CD7 and CD5 were also detected. During a process where stem cells are induced into γδ T cells, it is considered that the cells become positive for CD45, a marker of hematopoietic cells, and then positive for CD3, a marker of T cells, followed by the expression of γδ T cell markers TCRγ9 and TCRδ2. Therefore, the combinations of CD3 positive and CD8 positive, CD7 positive and CD5 negative, CD7 positive and CD5 positive, and CD45 positive, TCRγ9 positive, and TCRδ2 negative indicate cells that are in the process of being induced into γδ T cells.

### Comparative Example 1

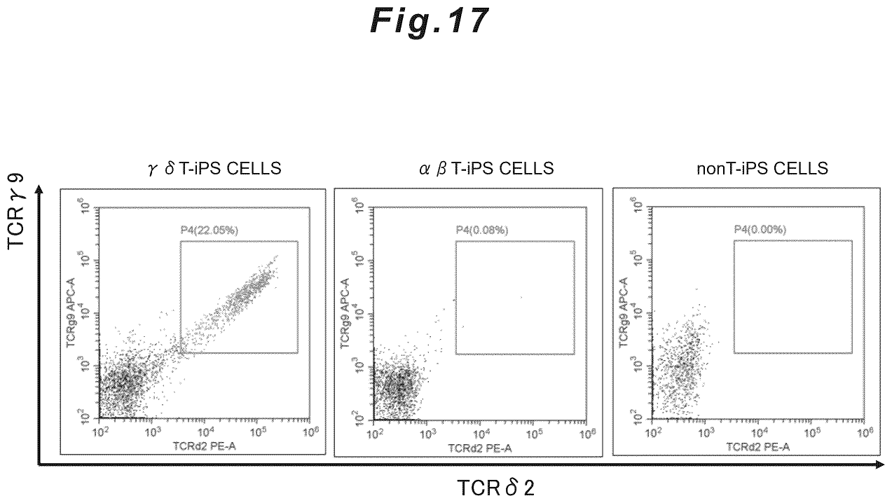

iPS cells induced from αβ T cells (αβ T-iPS cells) were prepared in a serum-free lymphocyte medium (X-VIVO 10, registered trademark, Lonza) supplemented with IL-2 and beads conjugated with anti-CD3 and anti-CD28 monoclonal antibodies on their surface (Dynabeads, registered trademark, Human T-Activator CD3/CD28, ThermoFisher). Using KLF4, OCT3/4, SOX2, and c-MYC, hematopoietic progenitor cells were induced from these αβ T-iPS cells in the same manner as in Example 8. Further, the hematopoietic progenitor cells were cultured for 3 weeks in the same manner as in Example 8. Flow cytometry analysis of the cells cultured for 3 weeks revealed that while they were CD45-positive, they were negative for TCRγ9 and TCR82, as shown in FIG. 17, indicating that γδ T cells were not induced.

### Comparative Example 2

iPS cells (nonT-iPS cells) induced from non-T cells lacking recombined TCR genes were prepared in a hematopoietic medium (StemSpan H3000, STEMCELL Technologies) supplemented with IL-6, SCF, TPO, FLT3 ligand, and IL-3, using KLF4, OCT3/4, SOX2, and c-MYC. Hematopoietic progenitor cells were induced from these nonT-iPS cells in the same manner as in Example 8, and the cells were further cultured for 3 weeks. Flow cytometry analysis of the cells cultured for 3 weeks showed that, although they were CD45-positive, they were negative for TCRγ9 and TCRδ2, as shown in FIG. 17, indicating that γδ T cells were not induced.

### Example 9

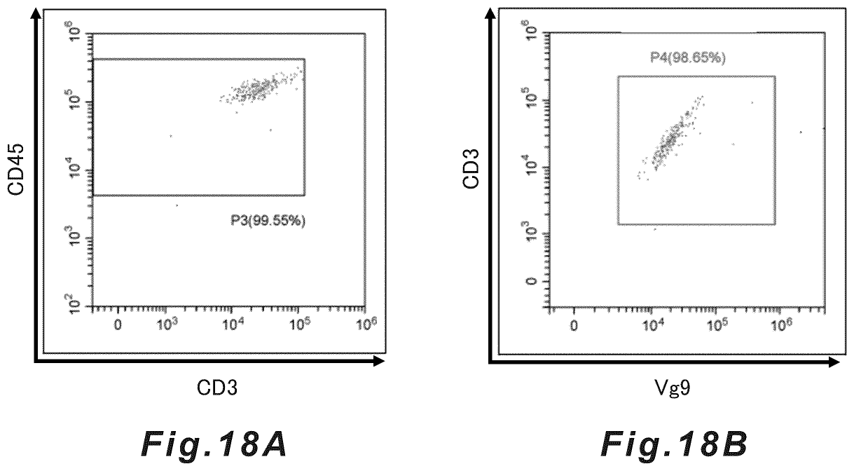

γδ T cells produced in Example 8 were collected and centrifuged to remove the supernatant. Next, the cells were resuspended in a differentiation medium supplemented with IL-7, IL-2, dexamethasone, and anti-CD3 (OKT3) antibody, and cultured at 37° C. in a 5% CO2 environment. After 3 days, the cells were collected, washed with PBS, centrifuged, and the supernatant was removed. The cells were then resuspended in a differentiation medium supplemented with IL-7, IL-2, and dexamethasone, and cultured at 37° C. in a 5% CO2 environment. After 4 days, mature γδ T cells were obtained. Flow cytometry analysis of the mature γδ T cells showed that, as depicted in FIG. 18, more than 98% of the cells were positive for both the combination of CD3 and CD45, as well as the combination of Vγ9 and CD3.

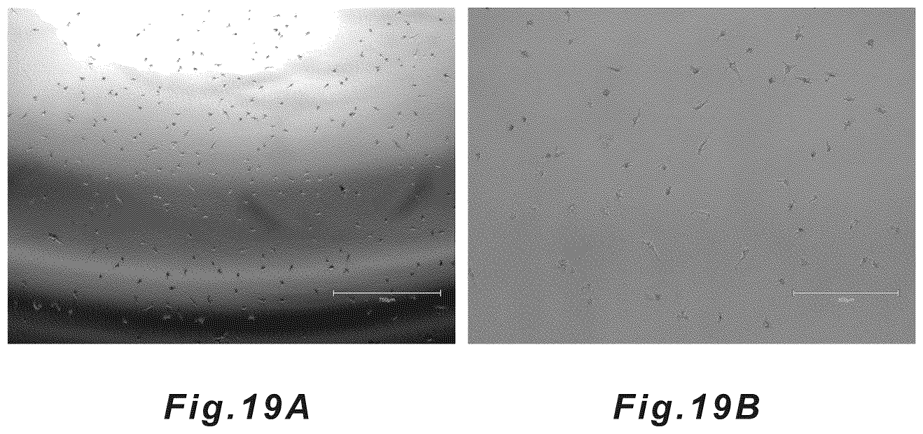

The mature γδ T cells were collected, centrifuged, and the supernatant was removed. The cells were then resuspended in a differentiation medium supplemented with IL-2, IL-4, IL-9, and IL-18, and seeded onto wells coated with anti-CD3 (OKT3) antibody and RetroNectin. The cells were cultured at 37° C. in a 5% CO2 environment. After 3 days, the cells were collected, washed with PBS, centrifuged, and the supernatant was removed. The cells were resuspended in a differentiation medium supplemented with IL-2, IL-4, IL-9, and IL-18, and cultured at 37° C. in a 5% CO2 environment. The medium was exchanged when its color changed, and the cells were passaged to larger wells when they covered the well bottom. This process was repeated to amplify the mature γδ T cells. As shown in FIG. 19, the morphology of the proliferating γδ T cells confirmed that they were activated by IL-2, IL-4, IL-9, IL-18, and anti-CD3 (OKT3) antibody.

1-20. (canceled)

21. A method for producing γδ T cells, comprising inducing differentiation of γδ T cells from pluripotent stem cells derived from T cells.

22. The method for producing γδ T cells according to claim 21, wherein said pluripotent stem cells are derived from γδ T cells.

23. The method for producing γδ T cells according to claim 21, wherein said γδ T cells induced from said pluripotent stem cells express γδ T cell receptors.

24. The method for producing γδ T cells according to claim 21, wherein said pluripotent stem cells comprise recombined γδ T cell receptor genes.

25. The method for producing γδ T cells according to claim 21, wherein said γδ T cells induced from said pluripotent stem cells express γδ T cell receptors.

26. The method for producing γδ T cells according to claim 21, wherein the inducing the differentiation of the γδ T cells from the pluripotent stem cells comprising:
inducing hematopoietic progenitor cells from said pluripotent stem cells, and inducing said γδ T cells from said hematopoietic progenitor cells.

27. γδ T cells induced from pluripotent stem cells derived from γδ T cells.

28. A pharmaceutical composition for cancer treatment, comprising γδ T cells induced from pluripotent stem cells derived from γδ T cells.
